(19) United States
(12) Patent Application Publication
(10) Pub. No.: US 2011/0002815 A1
(43) Pub. Date: Jan. 6, 2011

(54) FLUID PURIFIER WITH NON-LAMINAR FLOW STRUCTURE

(76) Inventors: Timothy N. Obee, South Windsor, CT (US); Wayde R. Schmidt, Pomfret Center, CT (US); Thomas H. Vanderspurt, Glastonbury, CT (US); Stephen O. Hay, Tolland, CT (US); Di Wei, Ellington, CT (US)

Correspondence Address: MARJAMA MULDOON BLASIAK & SULLIVAN LLP, 250 SOUTH CLINTON STREET, SUITE 300, SYRACUSE, NY, 13202, US

(21) Appl. No.: 12/667,292

(22) PCT Filed: Jul. 5, 2007
(86) PCT No.: PCT/US07/15583

§ 371 Date: Dec. 30, 2009

(51) Int. Cl. A61L 9/00; B05D 3/00; B01D 53/86

(52) U.S. Cl. 422/122; 427/299

(57) ABSTRACT

The substrate cell surfaces of a catalytic air purifier are so structured as to disrupt the occurrence of laminar flow along the flow path of the fluid passing therethrough. A plurality of substrates are connected in serial flow but axially offset relationship to obtain improved performance. Also, the dimensional aspects of the individually cells are selected so as to maintain adequate mass-transfer coefficient and UV photon penetration depths throughout the length thereof.

### BACKGROUND OF THE INVENTION

The present invention relates generally to air or fluid photocatalytic/thermocatalytic purifiers and, more particularly, to a purification system wherein the substrate to which the catalytic coating is applied is so structured and sized as to result in enhanced performance.

Indoor air can include trace amounts of contaminants, including carbon monoxide, ozone and volatile organic compounds such as formaldehyde, toluene, propanal, butene, and acetaldehyde. Adsorbent air filters, such as activated carbon, have been employed to remove these contaminants from the air. As air flows through the filter, the filter blocks the passage of the contaminants, allowing contaminant free air to flow from the filter. A drawback to employing filters is that they simply block the passage of contaminants and do not destroy them. Additionally, the filter is not effective in blocking ozone and carbon monoxide.

Titanium dioxide has been employed as a photocatalyst in an air purifier to destroy contaminants. When the titanium dioxide is illuminated with ultraviolet light, photons are absorbed by the titanium dioxide, promoting an electron from the valence band to the conduction band, thus producing a hole in the valence band and adding an electron in the conduction band. The promoted electron reacts with oxygen, and the hole remaining in the valence band reacts with water, forming reactive hydroxyl radicals. When a contaminant adsorbs onto the titanium dioxide catalyst, the hydroxyl radicals attack and oxidize the contaminants to water, carbon dioxide, and other substances.

Doped or metal oxide treated titanium dioxide can increase the effectiveness of the titanium dioxide photocatalyst. However, titanium dioxide and doped titanium dioxide are less effective or not effective in oxidizing carbon monoxide. Carbon monoxide (CO) is a colorless, odorless, and poisonous gas that is produced by the incomplete combustion of hydrocarbon fuels. Carbon monoxide is responsible for more deaths than any other poison and is especially dangerous in enclosed environments. Gold can be loaded on the titanium dioxide to act as an effective thermocatalyst for the room temperature oxidation of carbon monoxide to carbon dioxide.

Photocatalytically, titanium dioxide alone is less effective in decomposing ozone. Ozone (O.sub.3) is a pollutant that is released from equipment commonly found in the workplace, such as copiers, printer, scanners, etc. Ozone can cause nausea and headaches, and prolonged exposure to ozone can damage nasal mucous membranes, causing breathing problems. OSHA has set a permissible exposure limit (PEL) to ozone of 0.08 ppm over an eight hour period.

Ozone is a thermodynamically unstable molecule and decomposes very slowly up to temperatures of 250° C. At ambient temperatures, manganese oxide is effective in decomposing ozone by facilitating the oxidation of ozone to adsorbed surface oxygen atoms. These adsorbed oxygen atoms then combine with ozone to form an adsorbed peroxide species that desorbs as molecular oxygen.

Fluid purification systems have therefore been developed with catalytic coatings being applied to the surfaces of substrates over which the fluid is made to flow such that the catalyst oxidizes and decomposes the gaseous containments, including volatile organic compounds, carbon monoxide and ozone and that adsorb into the photocatalytic surface to form carbon dioxide, water, oxygen and other substances. In a photocatalyst based air purifier, gas-phase, including semi-volatile contaminants are destroyed by a photocatalyst. The photocatalyst itself is activated by photons of a suitable wavelength. The design of such a purifier brings both the contaminant and photon to the photocatalyst where oxidation of the contaminant can take place. To effectively accomplish this, the design must account for mass-transport of the contaminant and radiation transport of the photon. One possible support for the photocatalyst is a honeycomb monolith; walls of the honeycomb are coated with a thin layer of a photocatalyst. The honeycomb structure typically contains an array of equal sized “cells” or passages and is characterized by low pressure drop due to its unobstructed flow region and smooth walls. Arrays can also contain adjacent cells which have different cross sectional geometries or diameters. The typical dimensions of these substrates are such that the airflow through each passage of the honeycomb is laminar well before the exit of the honeycomb. This laminar flow places mass transfer limitations on reactor effectiveness.

When the associated flow regime is laminar, then mass transport of the contaminant to the catalyst is limited by molecular diffusion. For the situation in which the photocatalyst is sufficiently active the overall effectiveness of the contaminant destruction process will be limited by the molecular diffusion rate and, consequently, would be considered mass transport limited. There is thus a need to eliminate the occurrence of laminar flow within the length of the substrate so as to thereby increase the mass transfer efficiency of such a system.

In addition, and independent of this situation there is the “entrance length effect”. At the entrance to the honeycomb the mass transfer coefficient is greatest and decreases with distance in the flow direction, reaching a minimum when the velocity and contaminant fields are fully developed. The entrance length is the distance measured from the honeycomb entrance to the location of the fully developed regime (the fluid flow profile develops into a parabolic profile, i.e. laminar flow). The expression for the entrance length (L) is usually expressed in terms of the diameter (D) of a single honeycomb cell and is functionally related to the cell's Reynolds (Re_{D}) and Schmidt (Sc) numbers:

L/D=0.05Re_{D}*Sc

In the present case, we are considering that flow conditions are laminar if the Reynolds number value is less than 2000. A further constraint on the application of the honeycomb concept is the penetration depth of the UV photon into the interior of the honeycomb cell. The UV penetration depth is a geometry constraint and is independent of the flow velocity. Thus, the use of honeycombs can be limited in application to the UV penetration depth.

### SUMMARY OF THE INVENTION

Briefly, in accordance with one aspect of the invention, the substrate cell surface to which the catalytic coating is applied is so structured (e.g. textured) as to disrupt the occurrence of laminar flow and intentionally create turbulence along the flow path of the fluid passing therethrough.

In accordance with another aspect of the invention, the length of the substrate cells (X) is on the order of or less than the entrance length (L) such that the mass transfer limitations that are imposed by laminar flow that would otherwise occur are significantly mitigated.

In accordance with another aspect of the invention, rather than a single relatively long substrate, a plurality of relatively short substrates are placed in offset serial flow relationship.

In accordance with another aspect of the invention, textured features are introduced to create turbulence and reduce the occurrence of laminar flow along the surface of the substrate.

By yet another aspect of the invention, the substrate cells are so dimensioned as to maintain an adequate mass transfer coefficient along their length.

By still another aspect of the invention, the substrate is so dimensioned and structured as to maintain sufficient penetration depth at the photons throughout their lengths.

In the drawings as hereinafter described, a preferred embodiment is depicted; however, various other modifications and alternate constructions can be made thereto without departing from the spirit and scope of the invention.

### BRIEF DESCRIPTION OF THE DRAWINGS

FIG. 1 is a perspective view of a honeycomb substrate for a catalytic air cleaner.

FIG. 2 is a graphic illustration of the effect of a honeycomb length on its mass-transfer coefficient.

FIG. 3 is a graphic illustration of the entrance effect on the kinetic reaction rate of a honeycomb cell.

FIGS. 4A and 4B show representative example fluid flow profiles for cases when L<X and X<L, with single and segmented honeycomb arrays.

FIG. 5 is a schematic illustration of an offset combination of honeycomb structures.

FIG. 6 is an end view of a honeycomb cell with textured features added thereto.

FIGS. 7A, 7B and 7C show side views of a honeycomb array with a turbulator structure respectively offset and adjacent to the array.

FIGS. 8A, 8B and 8C show respective side views of segmented honeycomb arrays with and without gaps and with turbulator structures between individual segments.

### DETAILED DESCRIPTION OF THE INVENTION

A monolithic honeycomb cell array is shown at 11 in FIG. 1, comprising a plurality of integrally connected, multi-sided channels 12 extending in parallel relationship along the length X of the cell. A photocatalyst such as titanium dioxide is coated on the internal surface of the channels 12 and the coating is then illuminated with ultra violet (UV) light to cause a chemical reaction which tends to remove and destroy the contaminants of the air as the air is passed through the individual channels 12.

The effectiveness of the photocatalyst process will vary along the length X of the cell array 11 because of various factors including the entrance length effect, a variation in DV light penetration depth, and the tendency of the airflow becoming laminar in nature. Each of those effects will be discussed herein.

As the air enters the entrance to the individual cells 12, the mass-transfer coefficient is greatest at the entrance to the cells and decreases with distance in the flow direction, reaching a minimum when the velocity and contaminant fields are fully developed and have a generally parabolic profile. That distance measured from the honeycomb entrance (X=0) to the fully developed regime is referred to as the entrance length (L). If the diameter of the individual cells 12 is D, the entrance length (L) is usually expressed in terms of the individual honeycomb cell diameter D and is functionally related to the cell's Reynolds number (Re_{D}) and Schmidt number (Sc) as follows:

L/D=0.05Re_{D}*Sc.

When the physical length (X) of a honeycomb cell exceeds the entrance length (L), the overall mass transport is largely determined by the mass transport coefficient for the fully developed regime. That is, when L is less than X, the fully developed velocity (parabolic) profile develops within the length of the honeycomb so that the mass transfer coefficient (h₀) of the fully developed regime is within the honeycomb. This situation is a highly undesirable for system effectiveness.

On the other hand, if the physical length (X) of a honeycomb cell is less than the entrance length (L), then the flow profile never fully develops into a parabolic form within the honeycomb, in which the case h₀ is outside the honeycomb and causes the ratio of h/h₀ to get very large. The mass transfer coefficient, h, is now larger than h₀ for any location within the honeycomb flow passage. Then the overall mass transport coefficient is strongly dependent on the actual cell depth (X).

As shown in FIG. 2, the ratio of the mass transfer coefficient (h) to the mass transfer coefficient (h₀) of the fully developed region is shown. The overall mass transfer coefficient, in fact, is the integral of the local mass transfer coefficient (h) from the cell entrance (X=0) to the cell depth (X). Because of the logarithmic dependence, this integral is largely determined from the end point (X).

FIG. 2 is a graphic illustration of the effect of a honeycomb length (X) on the mass transfer coefficient (h). Here, the ordinate h/h₀ simply relates the new mass transport coefficient (h) to that of a fully established flow profile h₀. The abscissa, (X/D)/(Re*Sc), describes the interaction between the honeycomb geometry and the fluid flow conditions.

As will be seen, the curve decreases to a value of h/h₀ near the point where the abscissa, (X/D)/(Re*Sc) is equal to about 0.1. Thus, there is an opportunity to increase the mass transfer coefficient and thus enhance the performance of the purifier when the combination of honeycomb cell diameter and flow conditions render the abscissa of FIG. 2 to values less than about 0.1. As an example, the letter A₁ mocks a hypothetical case in which the initial length of the honeycomb and flow conditions results in a value of about 0.01 for the abscissa and a value of about 1.3 for the ordinate (i.e. ratio of mass transfer coefficients). Now if this honeycomb were divided (i.e. slashed perpendicular to the direction of the flow field) into two distinct pieces or sections of about equal lengths, then the physical depth of each section is also cut in half and, in turn, the location of the new point on the abscissa of FIG. 2 is reduced by half, as indicated by the letter A₂. If the two honeycomb sections are realigned but are offset by one half cell diameter, then the resulting mass transfer coefficient ratio would increase to about 1.7, as indicated by the letter A₂, for a net improvement of 1.3 (=1.7/1.3) in mass transport effect. As another example, if the honeycomb were divided into three distinct sections and each section realigned but offset by one half cell diameter, then the resulting mass-transfer coefficient ratio would increase to about 2.0 as indicated by A₃ for a net improvement of 1.5 (=2.0/1.3) in mass transport effect. These are examples demonstrating the potential benefit resulting from the segmented honeycomb concept.

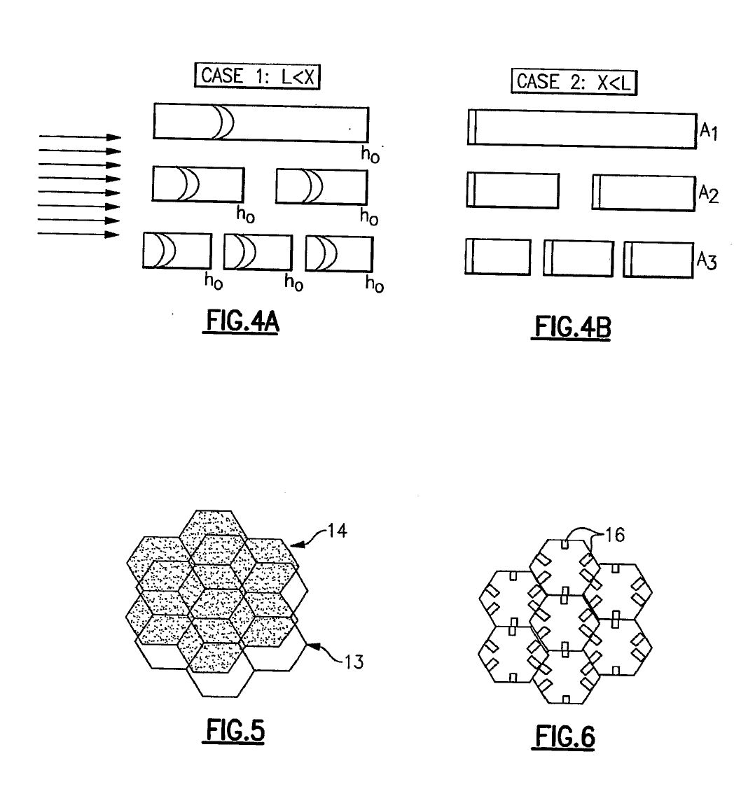

FIGS. 4A and 4B schematically show the flow field dependence for cases when L<X and when X<L when the honeycomb structure is segmented. In the former case, the flow fields have fully developed into parabolic profiles. In the latter more desired case, the parabolic flow profiles are established well outside the length of the honeycomb passages.

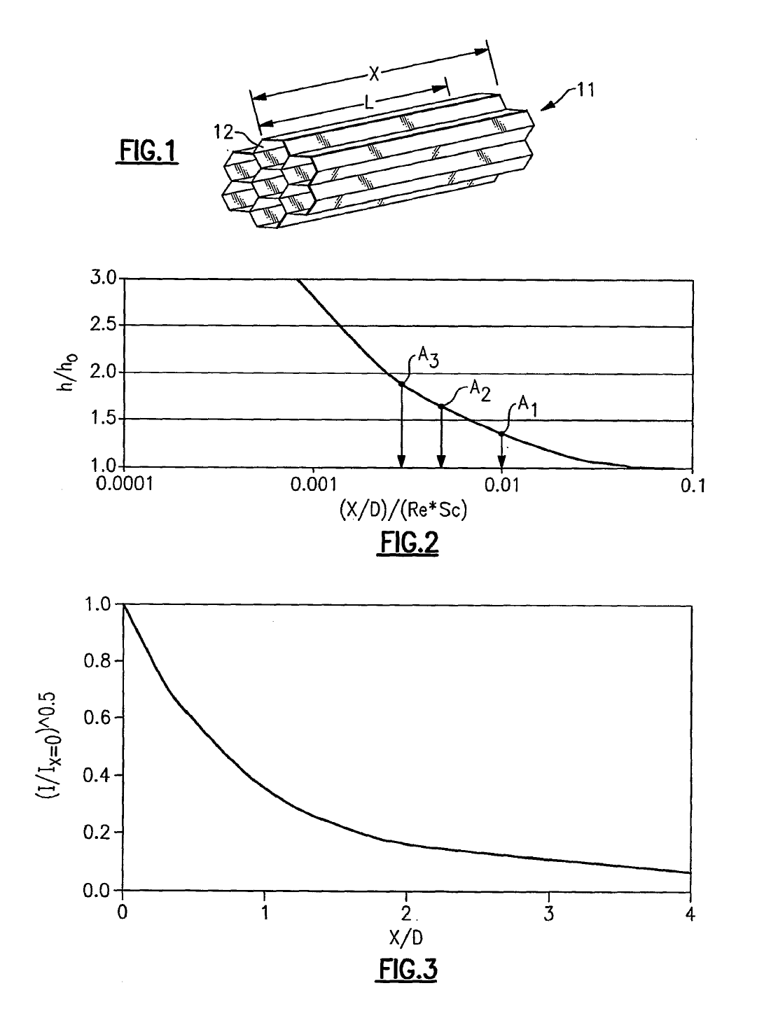

In addition to the mass transfer coefficient being affected by the dimensional features of a honeycomb based air purifier, the penetration depth of the UV photon in a honeycomb cell is also dependent on the dimensional features of the cell as shown in FIG. 3. Here, the UV flux ratio, the ordinate in FIG. 3, represents the ratio of kinetic oxidation rates on the photocatalyst and is shown as a function of the aspect ratio, X/D of the honeycomb cell.

The ordinate in FIG. 3 is the square root of the ratio of the UV flux at any cell depth (X) to the UV flux at the cell entrance, taken as X=0. The oxidation kinetics of the photocatalytic process is dependent on the UV flux raised to a power. In general, the power factor is dependent on the specific contaminant and further on the catalyst composition. In FIG. 3, square root dependence is assumed. For example, the photocatalyst titanium dioxide exhibits square root dependence for some contaminants.

As will be seen in FIG. 3, as the aspect ratio of the honeycomb structure, X/D, is increased beyond about 4, the UV flux ratio approaches 0. Accordingly, it is desirable to maintain the aspect ratio of the honeycomb cells at a value below about 4 and preferably at a value below about 2.

For the discussions above, it will be seen that both the mass transfer coefficient and the UV photon penetration depth are dependent on the aspect ratio X/D of the honeycomb cells. While it is desirable that the length X of the cell is less than the entrance length L, it is desirable to limit the cell length X such that the aspect ratio X/D is maintained within the parameters discussed hereinabove. On the other hand, the limiting of the cell length X may unnecessarily reduce the effective surface of the cell. Accordingly, as discussed hereinabove with respect to the FIG. 2 performance characteristics, rather than using a single honeycomb structure, it is desirable to use a plurality of shorter structures in an offset relationship such that, with very little pressure drop penalty, both the mass transfer coefficient and the photon utilization can be increased. Such an offset design is shown in FIG. 5 wherein a first honeycomb is shown at 13 and a second honeycomb 14 is placed in a downstream position and offset by a maximum distance D/2 in the radial direction from the axis of flow. The entrance end of the second honeycomb 14 is preferably placed in abutting relationship with the exit end of the first honeycomb 13. A third honeycomb (not shown) could then be placed in a similar offset relationship with the second honeycomb 14 but in axial alignment with the first honeycomb 13. Any number of honeycomb structures can then be used in series in this manner to achieve greater effectiveness of a honeycomb structure air purifier.

An alternate approach for improving the mass transport of contaminants is through the application of turbulators, protuberances or flow disruptors 16 as shown in FIG. 6. Such features can be internal to the passages (such a raised chevrons, turning vanes, trip strips, swirl features, guide vanes or other flow disruptors) or can be external to the passage (such as screens or meshes immediately adjacent to or offset from the face of the honeycomb array, but normal to the array axis) to create turbulence in the flow field that enters the honeycomb array. By another aspect of the invention, the plurality of substrates can be placed immediately adjacent to one another or offset by a gap, which may contain further flow disruptors. The disruptors 16 extend from the wall of the honeycomb and into the flow field to create Karman instability through vortex shedding. The shedding of vortices is, in effect, a turbulence generator which induces mixing and leads to the desired improved contaminant mass transport. To avoid a shadowing of the photocatalyst, the protuberances are preferably made of UV transparent material. Their location may be at the entrance of the honeycomb or at an intermediate location on the cell walls.

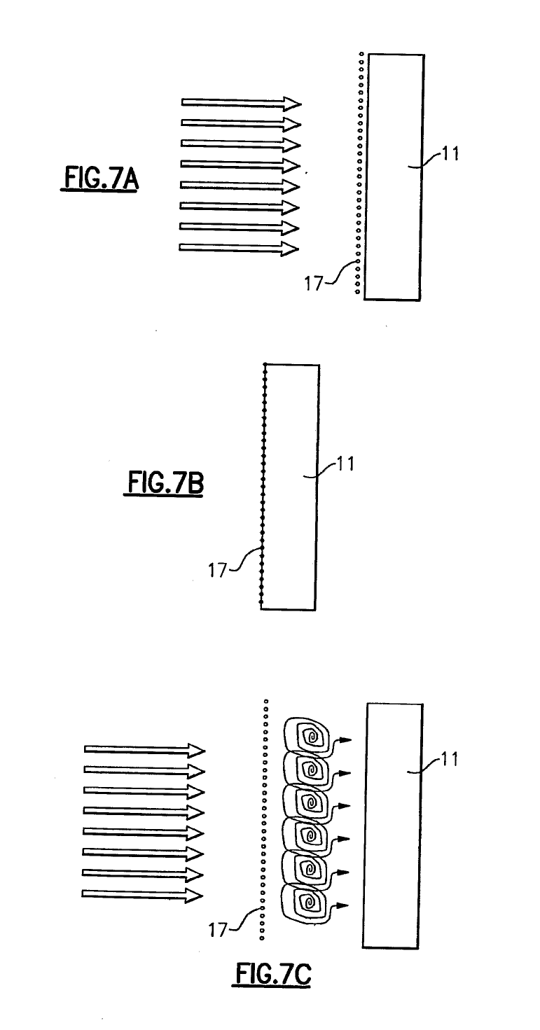

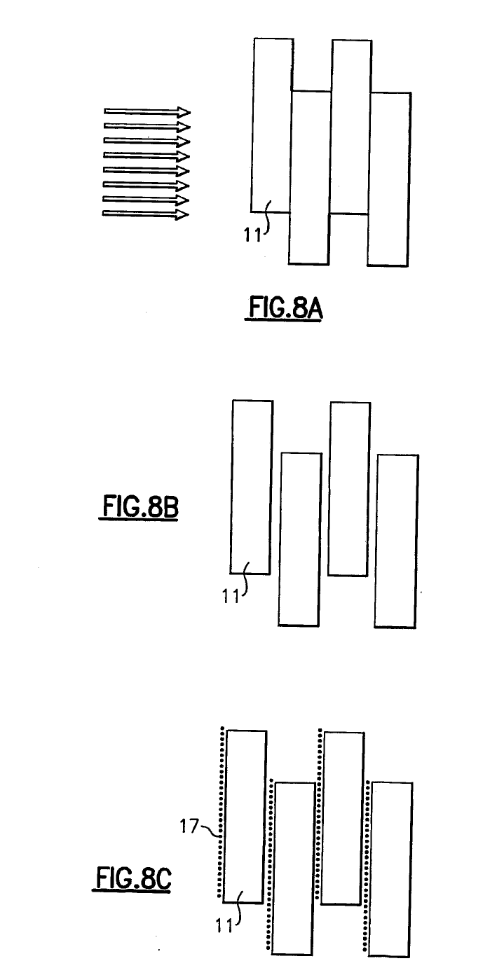

As another means of introducing protuberances at the honeycomb entrance, an interlaced grid 17 or mat-like construction (e.g. screen) can be positioned against the entrance face of the honeycomb as shown in FIGS. 7A and 7B. The screen 17 could be offset by a small distance sufficient to create and maintain turbulent flow fields downstream of the screen 17 as shown in FIG. 7C or be located immediately adjacent the entrance side to the honeycomb array as shown in FIG. 7B.

A combination of gaps between honeycomb segments 11 and turbulator structures is also contemplated to better tailor the flow field characteristics, with non limiting examples shown in FIGS. 8A, 8B and 8C. In FIG. 8A there are no gaps between the segments 11, in FIG. 8B gaps are provided between the segments 11, and in FIG. 8C interlaced grids 17 are placed in the gaps between segments 11.

Alternatively, a plurality of features can be formed on or in the surfaces of the honeycomb passages. To create Karman instability and vortex shedding, the protuberances must be aerodynamically blunt in the dimension perpendicular to the fluid velocity. An alternate means of causing mixing of the flow field is through swirl. For example, the protuberances could be designed in the shape of a turbine-blade so as to induce swirl. Alternate features such as, but not limited to, raised chevrons, turning vanes, trip strips, swirl features, guide vanes or other flow disruptors and combinations thereof, can be employed.

This latter concept offers the added benefit of an associated lower pressure drop.

We claim:

1. A purification system of the type having at least one substrate with a plurality of cells having surfaces extending along a flow path over which a contaminated fluid is intended to flow and at least one catalytic coating applied to said plurality of surfaces, wherein said substrate cells are so dimensioned and structured as to disrupt the occurrence of the laminar flow along said flow path.

2. A purification system as set forth in claim 1 wherein said substrate cells are so dimensioned that their lengths are equal to or less than an “entrance length” of the cells.

3. A purification system as set forth in claim 1 wherein said at least one substrate comprises a pair of serially connected substrates.

4. A purification system as set forth in claim 3 wherein said serially connected substrates have mutually engaged surfaces.

5. A purification system as set forth in claim 3 wherein said serially connected substrates are axially separate from mutual engagement.

6. A purification system as set forth in claim 3 wherein said serially connected substrates have a screen disposed therebetween.

7. A purification system as set forth in claim 3 wherein said pair of substrates are radially offset from one another.

8. A purification system as set forth in claim 1 wherein said substrate includes a plurality of protuberances on the cells surfaces to cause turbulence in the flow of the fluid thereover.

9. A purification system as set forth in claim 1 wherein said substrate is coated with a coating textured therein so as to promote turbulence within the flow stream of fluid passing thereover.

10. A purification system as set forth in claim 1 wherein a plurality of protuberances are provided at the upstream end of the substrate so as increase turbulence to the flow of fluid passing thereover.

11. A purification system as set forth in claim 1 wherein said substrate cell dimensions are such that:
(X/D)/(Re*Sc)<0.1
wherein:
X=the length of the substrate
D=the diameter of the substrate cells
Re=the Reynolds number of the substrate
Se=the Schmidt number of the substrate.

12. A purification system as set forth in claim 11 wherein:
(X/D)/(Re*Sc)<0.01.

13. A purification system as set forth in claim 1 wherein said substrate cell dimensions are such that:
X/D<4
wherein:
X=the length of the substrate
D=the diameter of the substrate cells.

14. A purification system as set forth in claim 13 wherein:
X/D<2.

15. A method of forming a purification system of the type having at least one substrate with a plurality of cells having surfaces extending along a flow path comprising the steps of:
forming a substrate with cells having surfaces which are so structured as to disrupt the occurrence of laminar flow of fluid along their lengths;
applying at least one catalytic coating on the surfaces thereof.

16. A method as set forth in claim 15 wherein the length of said substrate cells are equal to or less than the “entrance length” of the cells.

17. A method as set forth in claim 15 wherein said at least one substrate comprises a pair of substrates which are placed in serial flow relationship.

18. A method as set forth in claim 17 wherein said serially connected substrates have mutually engaged surfaces.

19. A method as set forth in claim 17 wherein said serially connected substrates are axially separate from mutual engagement.

20. A method as set forth in claim 17 wherein said serially connected substrates have a screen disposed therebetween.

21. A method as set forth in claim 17 wherein said pair of substrates are placed so as to be radially offset from one another.

22. A method as set forth in claim 15 and including the step of forming a plurality of protuberances on said cell surfaces.

23. A method as set forth in claim 15 and including the step of forming a plurality of protuberances on an upstream end of said substrate.

24. A method as set forth in claim 15 wherein said substrate cell dimensions are such that:
(X/D)/(Re*Sc)<0.1
wherein:
X=the length of the substrate
D=the diameter of the substrate cells
Re=the Reynolds number of the substrate
Sc=the Schmidt number of the substrate.

25. A method as set forth in claim 24 wherein:
(X/D)/(Re*Sc)<0.01.

26. A method as set forth in claim 15 wherein said substrate cell dimensions are such that:
X/D<4
wherein:
X=the length of the substrate
D=the diameter of the substrate cells.

27. A method as set forth in claim 26 wherein:
X/D<2.
